(19) United States
(12) Patent Application Publication
(10) Pub. No.: US 2018/0021079 A1
(43) Pub. Date: Jan. 25, 2018

(54) THERAPEUTIC ENERGY APPLYING STRUCTURE AND MEDICAL TREATMENT DEVICE

(71) Applicant: OLYMPUS CORPORATION, Tokyo (JP)

(72) Inventor: Yuta SUGIYAMA, Tokyo (JP)

(73) Assignee: OLYMPUS CORPORATION, Tokyo (JP)

(21) Appl. No.: 15/695,073

(22) Filed: Sep. 5, 2017

Related U.S. Application Data
(63) Continuation of application No. PCT/JP2015/061496, filed on Apr. 14, 2015.

(51) Int. Cl. A61B 18/08

(52) U.S. Cl. CPC A61B 18/082; A61B 2018/00095

(57) ABSTRACT

A therapeutic energy applying structure includes: an electric resistance pattern configured to generate heat by applying current; a heat transfer plate configured to transfer the heat from the electric resistance pattern to a body tissue; an adhesive layer having heat conductivity and provided between the electric resistance pattern and the heat transfer plate to adhesively fix the electric resistance pattern and the heat transfer plate; and a heat diffusion layer provided between the electric resistance pattern and the adhesive layer and configured to diffuse the heat from the electric resistance pattern and transfer the diffused heat to the adhesive layer. The adhesive layer is configured to adhesively fix the electric resistance pattern and the heat transfer plate via the heat diffusion layer.

### CROSS REFERENCES TO RELATED APPLICATIONS

This application is a continuation of PCT international application Ser. No. PCT/JP2015/061496, filed on Apr. 14, 2015 which designates the United States, incorporated herein by reference.

### 1. TECHNICAL FIELD

The disclosure relates to a therapeutic energy applying structure and a medical treatment device.

### 2. RELATED ART

Conventionally, medical treatment devices having a therapeutic energy applying structure to apply energy to body tissue for treatment (such as connection (or anastomosis) and dissection) have been known (see, for example, Japanese Patent Application Laid-Open No. 2014-124491).

The therapeutic energy applying structure described in Japanese Patent Application Laid-Open No. 2014-124491 includes a flexible substrate, a heat transfer plate, and an adhesive sheet described below.

The flexible substrate functions as a sheet heater. On one surface of the flexible substrate, an electric resistance pattern that generates heat by applying current is formed.

The heat transfer plate is made of a conductor such as copper. The heat transfer plate is disposed to face the one surface (electric resistance pattern) of the flexible substrate to contact body tissue and transfer heat from the electric resistance pattern to the body tissue (apply heat energy to the body tissue).

The adhesive sheet has good heat conductivity and insulation property, and is formed by mixing a ceramic having high thermal conductivity such as alumina or aluminum nitride with epoxy resin, for example. The adhesive sheet is provided between the flexible substrate and the heat transfer plate to adhesively fix the flexible substrate and the heat transfer plate.

### SUMMARY

In some embodiments, a therapeutic energy applying structure includes: an electric resistance pattern configured to generate heat by applying current; a heat transfer plate configured to transfer the heat from the electric resistance pattern to a body tissue; an adhesive layer having heat conductivity and provided between the electric resistance pattern and the heat transfer plate to adhesively fix the electric resistance pattern and the heat transfer plate; and a heat diffusion layer provided between the electric resistance pattern and the adhesive layer and configured to diffuse the heat from the electric resistance pattern and transfer the diffused heat to the adhesive layer. The adhesive layer is configured to adhesively fix the electric resistance pattern and the heat transfer plate via the heat diffusion layer.

In some embodiments, a medical treatment device includes the therapeutic energy applying structure.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic view illustrating a medical treatment system according to a first embodiment of the present invention;

FIG. 2 is an enlarged view of a distal end portion of a medical treatment device illustrated in FIG. 1;

FIG. 3 is a schematic view illustrating a therapeutic energy applying structure illustrated in FIG. 2;

FIG. 4 is a schematic view illustrating the therapeutic energy applying structure illustrated in FIG. 2;

FIG. 5 is a schematic view illustrating the therapeutic energy applying structure illustrated in FIG. 2;

FIG. 6 is a schematic view illustrating a therapeutic energy applying structure according to a second embodiment of the present invention;

FIG. 7 is a schematic view illustrating the therapeutic energy applying structure according to the second embodiment of the present invention;

FIG. 8 is a schematic view illustrating a therapeutic energy applying structure according to a third embodiment of the present invention;

FIG. 9 is a schematic view illustrating the therapeutic energy applying structure according to the third embodiment of the present invention;

FIG. 10 is a schematic view illustrating a therapeutic energy applying structure according to a fourth embodiment of the present invention;

FIG. 11 is a schematic view illustrating the therapeutic energy applying structure according to the fourth embodiment of the present invention;

FIG. 12 is a schematic view illustrating a therapeutic energy applying structure according to a fifth embodiment of the present invention; and

FIG. 13 is a schematic view illustrating the therapeutic energy applying structure according to the fifth embodiment of the present invention.

### DETAILED DESCRIPTION

Exemplary embodiments of the present invention will be described below with reference to the drawings. The present invention is not limited by the embodiments. The same reference signs are used to designate the same elements throughout the drawings.

Schematic Configuration of Medical Treatment System

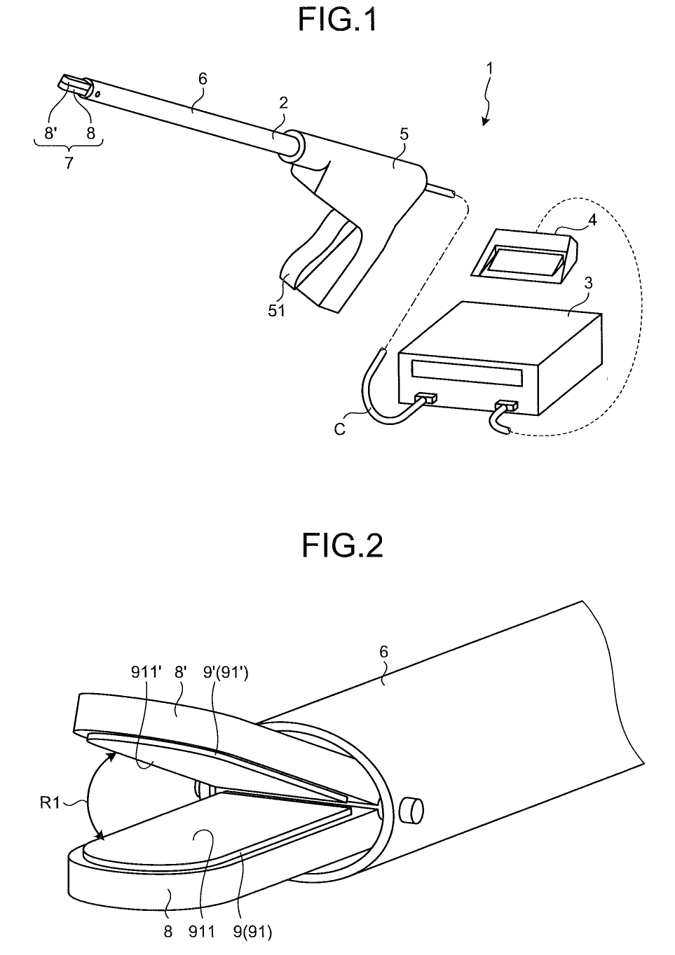

FIG. 1 is a schematic view illustrating a medical treatment system 1 according to a first embodiment of the present invention.

The medical treatment system 1 is configured to apply energy to body tissue as a target for treatment (such as connection (or anastomosis) and dissection). As illustrated in FIG. 1, the medical treatment system 1 includes a medical treatment device 2, a control device 3, and a foot switch 4.

Configuration of Medical Treatment Device

The medical treatment device 2 is, for example, a linear type surgical instrument for treating the body tissue through an abdominal wall. As illustrated in FIG. 1, the medical treatment device 2 includes a handle 5, a shaft 6, and a grasping portion 7.

The handle 5 is a part to be gripped by an operator. As illustrated in FIG. 1, the handle 5 is provided with an operation knob 51.

As illustrated in FIG. 1, the shaft 6 has a substantially cylindrical shape, and one end thereof is connected to the handle 5. Further, the grasping portion 7 is attached to the other end of the shaft 6. Inside the shaft 6, an opening and closing mechanism (not illustrated) is provided to open and close holding members 8 and 8′ (FIG. 1) constituting the grasping portion 7 in accordance with operation of the operation knob 51 by the operator. Further, inside the shaft 6, an electric cable C (FIG. 1) connected to the control device 3 is disposed from one end side to the other end side of the shaft 6 through the handle 5.

Configuration of Grasping Portion

FIG. 2 is an enlarged view of a distal end portion of the medical treatment device 2.

In FIGS. 1 and 2, an element indicated by a reference sign to which “′” is not added and an element indicated by a reference sign to which “′” is added are the same element. The same applies to subsequent drawings.

The grasping portion 7 is a portion for grasping the body tissue to treat the body tissue. As illustrated in FIG. 1 or 2, the grasping portion 7 includes a pair of holding members 8 and 8′.

The pair of holding members 8 and 8′ is axially supported at the other end of the shaft 6 so as to be opened/closed in a direction of an arrow R1 (FIG. 2) so that the body tissue can be grasped in accordance with the operation of the operation knob 51 by the operator.

As illustrated in FIG. 2, the holding members 8 and 8′ have therapeutic energy applying structures 9 and 9′, respectively.

Since the therapeutic energy applying structures 9 and 9′ have the same configuration, only the therapeutic energy applying structure 9 will be described below.

Configuration of Therapeutic Energy Applying Structure

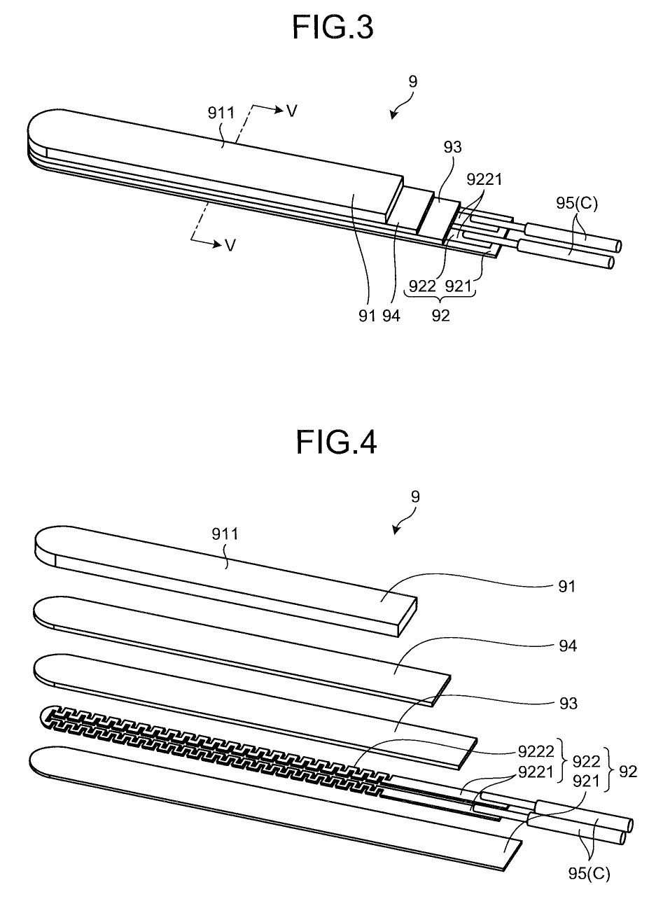

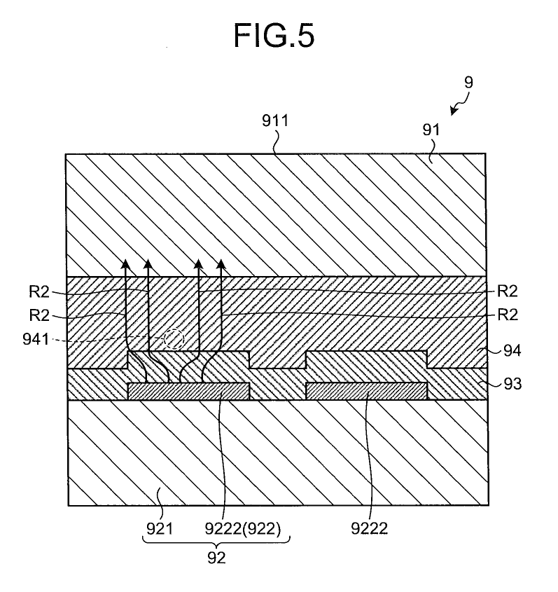

FIGS. 3 to 5 are views illustrating the therapeutic energy applying structure 9. Specifically, FIG. 3 is a perspective view of the therapeutic energy applying structure 9 from an upper side in FIG. 2. FIG. 4 is an exploded perspective view of FIG. 3. FIG. 5 is a cross-sectional view taken along a line V-V in FIG. 3.

The therapeutic energy applying structure 9 is attached to an upper side surface of the holding member 8 disposed on a lower side in FIGS. 1 and 2. The therapeutic energy applying structure 9 applies heat energy to the body tissue under control of the control device 3. As illustrated in FIGS. 3 to 5, the therapeutic energy applying structure 9 includes a heat transfer plate 91, a flexible substrate 92, a heat diffusion layer 93, an adhesive sheet (adhesive layer) 94, and two lead wires 95 (FIGS. 3 and 4).

The heat transfer plate 91 is an elongated thin plate made of a material such as copper, for example, and in a state where the therapeutic energy applying structure 9 is attached to the holding member 8, a treatment surface 911 as one plate surface faces a side of the holding member 8′ (the upper side in FIGS. 1 and 2). In addition, in a state where the body tissue is grasped by the holding members 8 and 8′, the treatment surface 911 of the heat transfer plate 91 contacts the body tissue to transfer heat from the flexible substrate 92 to the body tissue (applies heat energy to the body tissue).

A part of the flexible substrate 92 generates heat, and functions as a sheet heater that heats the heat transfer plate 91 by the generated heat. As illustrated in FIGS. 3 to 5, the flexible substrate 92 includes an insulating substrate 921 and a wiring pattern 922.

The insulating substrate 921 is an elongated sheet made of polyimide, which is an insulating material.

Here, a width of the insulating substrate 921 is set to be substantially the same as a width of the heat transfer plate 91. Further, a length of the insulating substrate 921 (a length in a horizontal direction in FIGS. 3 and 4) is set to be longer than a length of the heat transfer plate 91 (the length in the horizontal direction in FIGS. 3 and 4).

The wiring pattern 922 is obtained by processing stainless steel (SUS304), which is a conductive material, and is bonded to one surface of the insulating substrate 921 by thermocompression bonding. The wiring pattern 922 is used to heat the heat transfer plate 91. As illustrated in FIGS. 3 to 5, the wiring pattern 922 includes a pair of lead wire connecting portions 9221 (FIGS. 3 and 4) and an electric resistance pattern 9222 (FIGS. 4 and 5).

The material of the wiring pattern 922 is not limited to the stainless steel, and a conductive material such as platinum or tungsten may be used. Further, the wiring pattern 922 is not limited to a configuration in which the wiring pattern 922 is bonded to one surface of the insulating substrate 921 by thermocompression bonding, and a configuration in which the wiring pattern 922 is formed on the one surface by evaporation or the like may be used.

The pair of lead wire connecting portions 9221 is provided to extend from one end side (a right end side in FIGS. 3 and 4) toward the other end side (a left end side in FIGS. 3 and 4) of the insulating substrate 921, facing each other along a width direction of the insulating substrate 921. Each of the two lead wires 95 (FIGS. 3 and 4) constituting the electric cable C is joined (connected) to each of the pair of lead wire connecting portions 9221.

The electric resistance pattern 9222 has one end connected (by conduction) to one of the lead wire connecting portions 9221 and has the other end connected (by conduction) to the other of the lead wire connecting portions 9221 so as to be formed into a U shape along an outer edge of the insulating substrate 921. The electric resistance pattern 9222 generates heat by applying voltage (by applying current) to the pair of lead wire connecting portions 9221 by the control device 3 via the two lead wires 95.

The heat diffusion layer 93 is formed by applying energy to materials in a particulate state of several hundred microns or smaller, a molecular state, or an atomic state, and formed on one surface of the flexible substrate (a surface on a side of the wiring pattern 922) so that a part of the pair of lead wire connecting portions 9221 is exposed (FIGS. 3 and 4). The heat diffusion layer 93 is connected to the electric resistance pattern 9222 so as to be capable of transferring heat and diffuses heat from the electric resistance pattern 9222.

As illustrated in FIGS. 3 to 5, the adhesive sheet (adhesive layer) 94 is provided between the heat transfer plate 91 and the flexible substrate 92 on which the heat diffusion layer 93 is formed. In a state where a part of the flexible substrate 92 protrudes from the heat transfer plate 91, a surface of the heat transfer plate 91 opposite to the treatment surface 911 and the one surface of the flexible substrate 92 (the surface on the side of the wiring pattern 922 and the heat diffusion layer 93) are adhesively fixed. The adhesive sheet 94 is an elongated sheet having good heat conductivity and insulation property, withstanding high temperature, and having adhesive property, and formed by mixing a highly heat conductive filler such as alumina, boron nitride, graphite or aluminum with a resin such as epoxy or polyurethane, for example.

Here, a width of the adhesive sheet 94 is set to be substantially the same as widths of the heat transfer plate 91 and the insulating substrate 921. Further, a length of the adhesive sheet 94 (the length in the horizontal direction in FIGS. 3 and 4) is set to be longer than the length of the heat transfer plate 91 (the length in the horizontal direction in FIGS. 3 and 4), and shorter than the length of the insulating substrate 921 (the length in the horizontal direction in FIGS. 3 and 4).

Material and Thickness of Heat Diffusion Layer

In the first embodiment, a material having thermal conductivity of 2.5 [W/(m·K)] and a coefficient of thermal expansion of 75 [ppm/° C.] at or above a glass transition temperature is used as the adhesive sheet 94. Further, a thickness of the adhesive sheet 94 is set to 50 [μm].

Further, in the first embodiment, a coefficient of thermal expansion of the wiring pattern 922 (stainless steel (SUS304)) is 17 [ppm/° C.].

A material that satisfies following first to third conditions is used as the heat diffusion layer 93, and a thickness of the heat diffusion layer 93 is set to satisfy the second condition.

The first condition is that thermal conductivity of the heat diffusion layer 93 is higher than the thermal conductivity of the adhesive sheet 94.

The second condition is that thermal resistance per unit cross-sectional area of the heat diffusion layer 93 is smaller than thermal resistance per unit cross-sectional area of the adhesive sheet 94.

The thermal resistance per unit cross-sectional area of the heat diffusion layer 93 is given by T1/α1 in which a thickness of the heat diffusion layer 93 is denoted by T1 and the thermal conductivity of the heat diffusion layer 93 is denoted by α1. The thermal resistance per unit cross-sectional area of the adhesive sheet 94 is given by T2/α2 in which the thickness of the adhesive sheet 94 is denoted by T2 (50 [μm]) and the thermal conductivity of the adhesive sheet 94 is denoted by α2 (2.5 [W/(m·K)].

The third condition is that a coefficient of thermal expansion of the heat diffusion layer 93 is closer to a coefficient of thermal expansion of the wiring pattern 922 than a coefficient of thermal expansion of the adhesive sheet 94.

Specifically, in the first embodiment, a diamond-like carbon (DLC) film formed on the one surface (the surface on the side of the wiring pattern 922) of the flexible substrate 92 by chemical vapor deposition (CVD) is used as the heat diffusion layer 93.

Thermal conductivity of DLC is 8 [W/(m·K)]. Thus the thermal conductivity of DLC satisfies the first condition (higher than the thermal conductivity of 2.5 [W/(m·K)] of the adhesive sheet 94). Further, a coefficient of thermal expansion of DLC is 5 [ppm/° C.]. Thus the coefficient of thermal expansion of DLC satisfies the third condition (closer to the coefficient of thermal expansion (17 [ppm/° C.]) of the wiring pattern 922 than the coefficient of thermal expansion of 75 [ppm/° C.] of the adhesive sheet 94).

Further, in the first embodiment, the thickness T1 of the heat diffusion layer 93 is set to 10 [μm]. That is, the thermal resistance per unit cross-sectional area of the heat diffusion layer 93 (T1 (10 [μm])/α1 (8 [W/(m·K)])) is smaller than the thermal resistance per unit cross-sectional area of the adhesive sheet 94 (T2 (50 [μm])/α2 (2.5 [W/(m·K)])), and satisfies the second condition.

As long as the first to third conditions mentioned above are satisfied, the heat diffusion layer 93 is not limited to the DLC film (an amorphous film made of a carbon allotrope). Diamond, alumina of a highly heat conductive ceramic, aluminum nitride, silicon nitride, or silica may be used. The method of forming the heat diffusion layer 93 is not limited to CVD as long as the layer is formed by application of energy to materials in a particulate state of several hundred microns or smaller, a molecular state, or an atomic state. The heat diffusion layer 93 may be formed by physical vapor deposition (PVD), sputtering, thermal spraying, an aerosol deposition method, or plating.

Configurations of Control Device and Foot Switch

The foot switch 4 is a portion operated by a foot of the operator. Switching between on and off to apply current from the control device 3 to the medical treatment device 2 (the electric resistance pattern 9222) is performed in accordance with the operation of the foot switch 4.

Means for switching on and off as described above is not limited to the foot switch 4, and other means such as a switch operated manually may also be adopted.

The control device 3 is configured to include a central processing unit (CPU) and the like and comprehensively controls operation of the medical treatment device 2 according to a predetermined control program. More specifically, the control device 3 applies a voltage to the electric resistance pattern 9222 via the electric cable C (the two lead wires 95) in accordance with the operation of the foot switch 4 (the operation of turning on to apply current) by the operator to heat the heat transfer plate 91.

Operation of Medical Treatment Device

Next, operation (an operation method) of the medical treatment system 1 mentioned above will be described.

The operator grips the medical treatment device 2 and inserts the distal end portion (the grasping portion 7 and a part of the shaft 6) of the medical treatment device 2 into the abdominal cavity through an abdominal wall using a trocar or the like, for example. Further, the operator operates the operation knob 51 and grasps body tissue to be treated by the holding members 8 and 8′.

Next, the operator operates the foot switch 4 to switch on to apply current from the control device 3 to the medical treatment device 2. When the switch is on, the control device 3 applies a voltage to the wiring pattern 922 via the electric cable C (the two lead wires 95) to heat the heat transfer plate 91. Then, the body tissue in contact with the heat transfer plate 91 is treated by the heat of the heat transfer plate 91.

The therapeutic energy applying structure 9 according to the present embodiment described above includes the heat diffusion layer 93 that is connected to the electric resistance pattern 9222 so as to be capable of transferring heat and diffuses heat from the electric resistance pattern 9222.

Therefore, for example, even if a resin component included in the adhesive sheet 94 is deteriorated and vaporized by heat, and a highly heat insulating portion 941 such as bubbles having high heat insulation performance is generated in the adhesive sheet 94 as illustrated in FIG. 5, a portion of the electric resistance pattern 9222 close to the highly heat insulating portion 941 is not locally overheated.

Specifically, as indicated by an arrow R2 in FIG. 5, the heat from the portion of the electric resistance pattern 9222 close to the highly heat insulating portion 941 is once diffused by the heat diffusion layer 93. Thereafter, the heat is transferred to the heat transfer plate 91 via the adhesive sheet 94 so as to avoid the highly heat insulating portion 941.

In particular, the heat diffusion layer 93 is made of a material and has a thickness that satisfy the first and second conditions (the thermal conductivity and the thermal resistance in relation to the adhesive sheet 94).

Therefore, after the heat from the portion of the electric resistance pattern 9222 close to the highly heat insulating portion 941 is effectively diffused by the heat diffusion layer 93, the heat can be satisfactorily transferred to the heat transfer plate 91 via the adhesive sheet 94 so as to avoid the highly heat insulating portion 941.

Therefore, the therapeutic energy applying structure 9 according to the present embodiment has an effect of avoiding disconnection of the electric resistance pattern 9222 due to local overheat.

Further, in the therapeutic energy applying structure 9 according to the present embodiment, the heat diffusion layer 93 is provided between the flexible substrate 92 (the wiring pattern 922) and the adhesive sheet 94.

Therefore, even if the highly heat insulating portion 941 is generated in the adhesive sheet 94, as indicated by the arrow R2 in FIG. 5, a heat transfer path from the electric resistance pattern 9222 to the heat transfer plate 91 can be sufficiently secured.

In a conventional configuration, the adhesive sheet is adhesively fixed to the electric resistance pattern by a mechanical anchor effect. In such a fixed state, a part of the adhesive sheet may peel off from the electric resistance pattern in some cases. In such a case, a portion from which the adhesive sheet peeled off (a gap between the electric resistance pattern and the adhesive sheet) serves as an air layer having high heat insulation performance, which is incapable of transferring the heat from the electric resistance pattern. That is, also in a case where a part of the adhesive sheet is peeled off from the electric resistance pattern, a situation similar to a case where the part is deteriorated and vaporized arises.

In contrast, in the therapeutic energy applying structure 9 according to the present embodiment, the heat diffusion layer 93 is formed on the one surface of the flexible substrate 92 (the surface on the side of the wiring pattern 922) by application of energy to materials in a particulate state, a molecular state, or an atomic state.

Therefore, adhesion force between the electric resistance pattern 9222 and the heat diffusion layer 93 can be made higher than adhesion force between the electric resistance pattern and the adhesive sheet in the conventional configuration. That is, the heat diffusion layer 93 is hardly peeled off from the electric resistance pattern 9222. Therefore, even in consideration of peeling off of the heat diffusion layer 93 from the electric resistance pattern 9222, local overheat and disconnection of the electric resistance pattern 9222 can be avoided.

In particular, the heat diffusion layer 93 is made of a material that satisfies the third condition (the coefficient of thermal expansion in relation to the adhesive sheet 94 and the wiring pattern 922).

Therefore, expansion and contraction of the heat diffusion layer 93 can conform to expansion and contraction of the wiring pattern 922 in accordance with temperature change, which makes it difficult for the heat diffusion layer 93 to peel off from the electric resistance pattern 9222.

### Second Embodiment

Next, a second embodiment of the present invention will be described.

In the following description, the same reference signs are used to designate the same elements as those in the first embodiment, and detailed explanation thereof will be omitted or simplified.

A medical treatment system according to the second embodiment is different from the medical treatment system 1 described in the first embodiment mentioned above in the configuration of the therapeutic energy applying structures 9 and 9′. In the second embodiment, each therapeutic energy applying structure provided in each of the holding members 8 and 8′ has the same configuration. Therefore, only the therapeutic energy applying structure provided in the holding member 8 will be described below.

Configuration of Therapeutic Energy Applying Structure

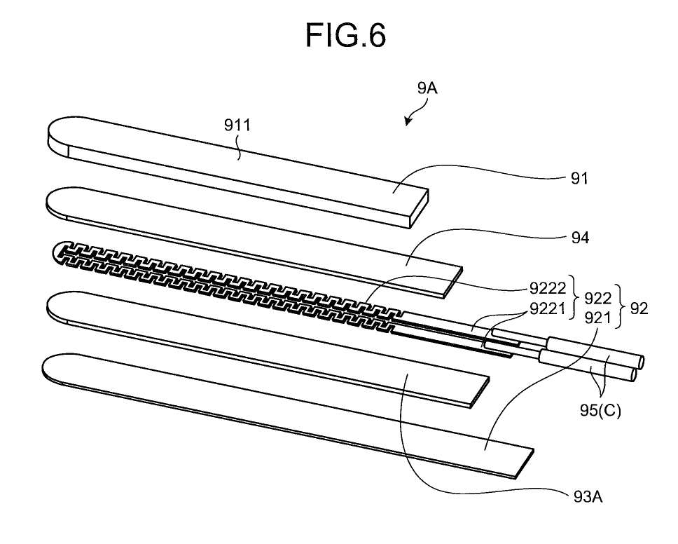

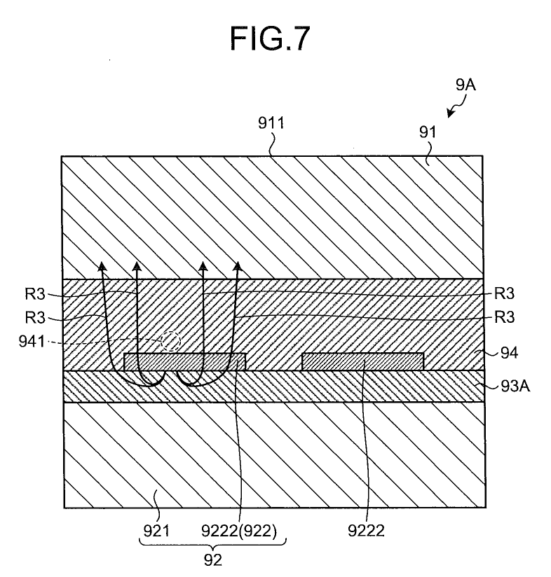

FIGS. 6 and 7 are views illustrating a therapeutic energy applying structure 9A according to the second embodiment of the present invention. Specifically, FIG. 6 is an exploded perspective view corresponding to FIG. 4. Further, FIG. 7 is a cross-sectional view corresponding to FIG. 5.

As illustrated in FIG. 6 or 7, the therapeutic energy applying structure 9A according to the second embodiment has a heat diffusion layer 93A instead of the heat diffusion layer 93 of the therapeutic energy applying structure 9 (FIGS. 3 to 5) described in the first embodiment mentioned above.

Specifically, similar to the heat diffusion layer 93 described in the first embodiment mentioned above, the heat diffusion layer 93A is formed by application of energy to materials in a particulate state of several hundred microns or smaller, a molecular state, or an atomic state, and is formed between the insulating substrate 921 and the wiring pattern 922 as illustrated in FIG. 6 or 7.

Similar to the heat diffusion layer 93 described in the first embodiment mentioned above, a material and a thickness of the heat diffusion layer 93A are set so as to satisfy the first to third conditions.

Even if the heat diffusion layer 93A is used instead of the heat diffusion layer 93 as in the second embodiment described above, as indicated by an arrow R3 in FIG. 7, heat from the portion of the electric resistance pattern 9222 close to the highly heat insulating portion 941 is once diffused by the heat diffusion layer 93A, and then, the heat can be transferred to the heat transfer plate 91 via the wiring pattern 922 or the adhesive sheet 94 so as to avoid the highly heat insulating portion 941. Therefore, effects similar to those of the first embodiment mentioned above are obtained.

### Third Embodiment

Next, a third embodiment of the present invention will be described.

In the following description, the same reference signs are used to designate the same elements as those in the first embodiment, and detailed explanation thereof will be omitted or simplified.

A medical treatment system according to the third embodiment is different from the medical treatment system 1 described in the first embodiment mentioned above in the configuration of the therapeutic energy applying structures 9 and 9′. In the third embodiment, each therapeutic energy applying structure provided in each of the holding members 8 and 8′ has the same configuration. Therefore, only the therapeutic energy applying structure provided in the holding member 8 will be described below.

Configuration of Therapeutic Energy Applying Structure

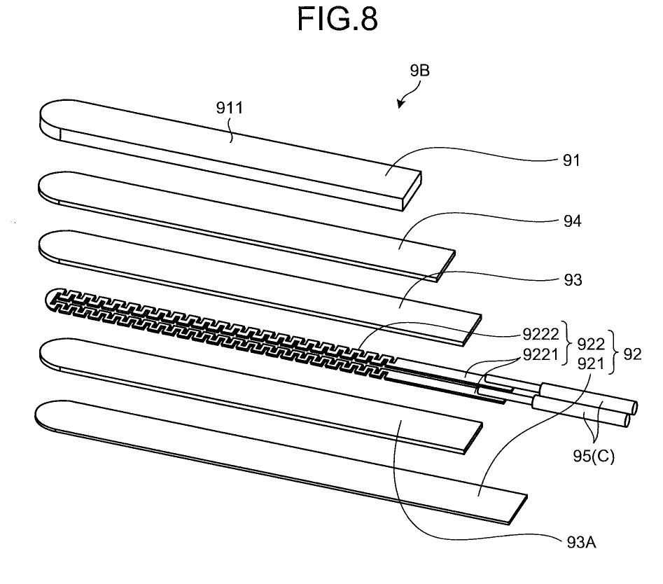

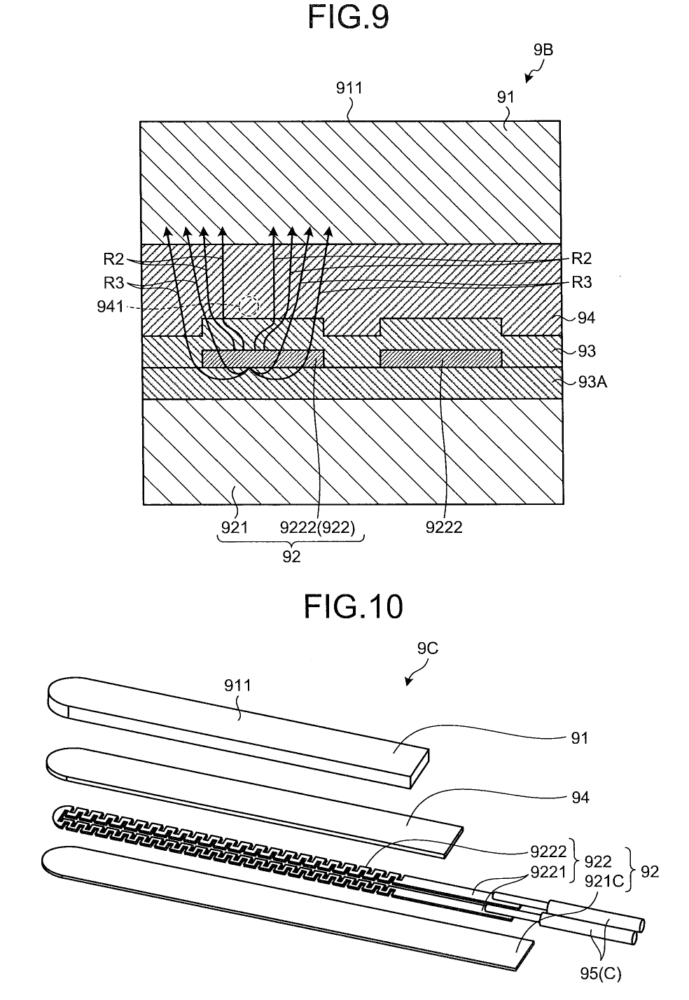

FIGS. 8 and 9 are views illustrating a therapeutic energy applying structure 9B according to the third embodiment of the present invention. Specifically, FIG. 8 is an exploded perspective view corresponding to FIG. 4. Further, FIG. 9 is a cross-sectional view corresponding to FIG. 5.

As illustrated in FIG. 8 or 9, in the therapeutic energy applying structure 9B according to the third embodiment, the heat diffusion layer 93A described in the second embodiment mentioned above is added to the therapeutic energy applying structure 9 (FIGS. 3 to 5) described in the first embodiment mentioned above. That is, in the therapeutic energy applying structure 9B according to the third embodiment, the two heat diffusion layers 93 and 93A, which are provided independently of each other, are employed.

The two heat diffusion layers 93 and 93A may be made of the same material and have the same thickness as long as the first to third conditions described in the first embodiment mentioned above are satisfied, or may be made of different materials and have different thicknesses.

Even if the two heat diffusion layers 93 and 93A, which are provided independently of each other, are employed as in the third embodiment described above, heat from the portion of the electric resistance pattern 9222 close to the highly heat insulating portion 941 can be transferred to the heat transfer plate 91 following a heat transfer path indicated by the arrows R2 and R3 in FIG. 9. Therefore, effects similar to those of the first and second embodiments mentioned above are obtained.

### Fourth Embodiment

Next, a fourth embodiment of the present invention will be described.

In the following description, the same reference signs are used to designate the same elements as those in the first embodiment, and detailed explanation thereof will be omitted or simplified.

A medical treatment system according to the fourth embodiment is different from the medical treatment system 1 described in the first embodiment mentioned above in the configuration of the therapeutic energy applying structures 9 and 9′. In the fourth embodiment, each therapeutic energy applying structure provided in each of the holding members 8 and 8′ has the same configuration. Therefore, only the therapeutic energy applying structure provided in the holding member 8 will be described below.

Configuration of Therapeutic Energy Applying Structure

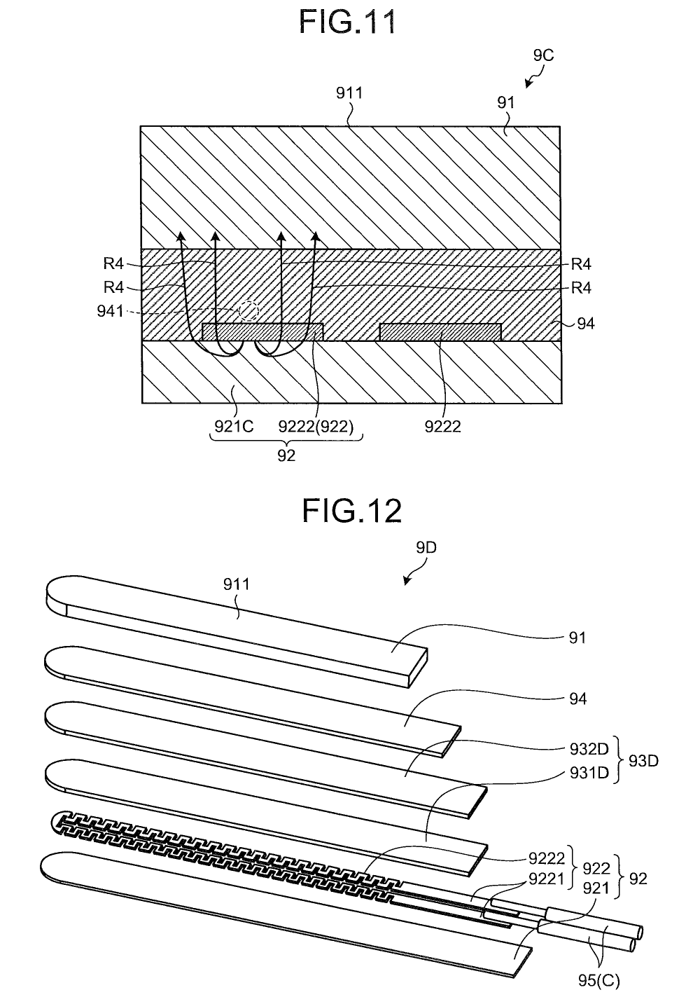

FIGS. 10 and 11 are views illustrating a therapeutic energy applying structure 9C according to the fourth embodiment of the present invention. Specifically, FIG. 10 is an exploded perspective view corresponding to FIG. 4. FIG. 11 is a cross-sectional view corresponding to FIG. 5.

As illustrated in FIG. 10 or 11, the therapeutic energy applying structure 9C according to the fourth embodiment does not have the heat diffusion layer 93 of the therapeutic energy applying structure 9 (FIGS. 3 to 5) described in the first embodiment mentioned above. Instead, the therapeutic energy applying structure 9C has an insulating substrate 921C whose material and thickness are different from those of the insulating substrate 921 (polyimide).

Specifically, the insulating substrate 921C has a material and a thickness that satisfy the first to third conditions described in the first embodiment mentioned above so as to have a function of the heat diffusion layer according to the present invention.

Here, as the material of the insulating substrate 921C, for example, a highly heat resistant insulating material such as aluminum nitride, alumina, glass, or zirconia can be used.

Even if the heat diffusion layer 93 is not provided and the insulating substrate 921C functions as the heat diffusion layer as in the fourth embodiment described above, as indicated by an arrow R4 in FIG. 11, heat from the portion of the electric resistance pattern 9222 close to the highly heat insulating portion 941 is once diffused by the insulating substrate 921C, and then, the heat can be transferred to the heat transfer plate 91 via the wiring pattern 922 or the adhesive sheet 94 so as to avoid the highly heat insulating portion 941. Therefore, effects similar to those of the first embodiment mentioned above are obtained.

### Modification of Fourth Embodiment

In the therapeutic energy applying structures 9 (9′), 9A, and 9B described in the first to third embodiments mentioned above, the insulating substrate 921C described in the fourth embodiment mentioned above may be used instead of the insulating substrate 921.

### Fifth Embodiment

Next, a fifth embodiment of the present invention will be described.

In the following description, the same reference signs are used to designate the same elements as those in the first embodiment, and detailed explanation thereof will be omitted or simplified.

A medical treatment system according to the fifth embodiment is different from the medical treatment system 1 described in the first embodiment mentioned above in the configuration of the therapeutic energy applying structures 9 and 9′. In the fifth embodiment, each therapeutic energy applying structure provided in each of the holding members 8 and 8′ has the same configuration. Therefore, only the therapeutic energy applying structure provided in the holding member 8 will be described below.

Configuration of Therapeutic Energy Applying Structure

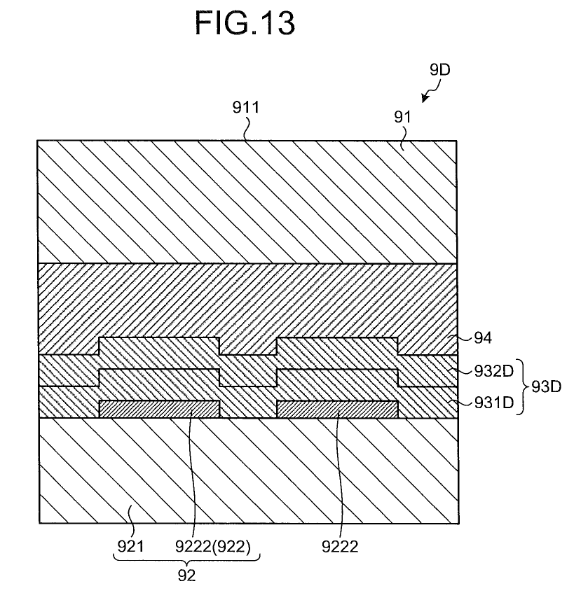

FIGS. 12 and 13 are views illustrating a therapeutic energy applying structure 9D according to the fifth embodiment of the present invention. Specifically, FIG. 12 is an exploded perspective view corresponding to FIG. 4. Further, FIG. 13 is a cross-sectional view corresponding to FIG. 5.

As illustrated in FIG. 12 or 13, in the therapeutic energy applying structure 9D according to the fifth embodiment, a heat diffusion layer 93D is used instead of the heat diffusion layer 93 in the therapeutic energy applying structure 9 (FIGS. 3 to 5) described in the first embodiment mentioned above.

Specifically, similar to the heat diffusion layer 93 described in the first embodiment mentioned above, the heat diffusion layer 93D is formed by application of energy to materials in a particulate state of several hundred microns or smaller, a molecular state, or an atomic state, and includes an insulating layer 931D and a heat conducting layer 932D which are provided independently of each other, as illustrated in FIG. 12 or 13.

The insulating layer 931D is formed on the one surface (the surface on the wiring pattern 922 side) of the flexible substrate 92.

The heat conducting layer 932D is formed on the insulating layer 931D.

Similar to the heat diffusion layer 93 described in the first embodiment mentioned above, the insulating layer 931D and the heat conducting layer 932D have a material and a thickness that satisfy the first to third conditions.

For example, as the material of the insulating layer 931D, an inorganic material having an insulation property is preferable, and silica, yttria, alumina, barium titanate, or the like can be used. Further, as the material of the heat conducting layer 932D, a material having high thermal conductivity, for example, nickel, gold, tin, a nickel tungsten alloy or the like which can be formed by electroless plating can be used. The heat conducting layer 932D is not limited to the material that can be formed by the electroless plating, and a conductive material that can be formed by evaporation, sputtering, or the like may be used.

According to the fifth embodiment described above, there are the following effects in addition to effects similar to those of the first embodiment mentioned above.

For example, assume that the material of the insulating layer 931D is silica (thermal conductivity: 10 [W/(m·K)]) and the material of the heat conducting layer 932D is nickel (thermal conductivity: 90 [W/(m·K)]). Further, a thickness of the insulating layer 931D is set to 1 [μm] and a thickness of the heat conducting layer 932D is set to 10 [μm], so that the thicknesses are substantially the same as that of the heat diffusion layer 93 described in the first embodiment mentioned above.

In the above design, thermal resistance per unit cross-sectional area of the entire heat diffusion layer 93D (1 [μm]/10 [W/(m·K)]+10 [μm]/90 [W/(m·K)]) can be set to an extremely small value compared to the thermal resistance per unit cross-sectional area of the heat diffusion layer 93 (10 [μm]/8 [W/(m·K)]) formed of a single layer of the DLC film described in the first embodiment mentioned above. Therefore, the effect of the first embodiment mentioned above can be suitably realized. Further, since the thermal resistance per unit cross-sectional area of the entire heat diffusion layer 93D is a relatively small value, the degree of freedom of each of the thicknesses of the insulating layer 931D and the heat conducting layer 932D can be improved in a case where the thicknesses are set to satisfy the second condition (the thermal resistance of the heat diffusion layer 93D in relation to the thermal resistance of the adhesive sheet 94).

### Modification of Fifth Embodiment

In the fifth embodiment mentioned above, the insulating layer 931D is formed of a single layer. Alternatively, the insulating layer 931D may be formed of two or more layers provided independently of one another. Similarly, the heat conducting layer 932D may be formed of two or more layers provided independently of one another.

### Other Embodiments

Although the modes for carrying out the present invention has been described so far, the present invention should not be limited only by the first to fifth embodiments mentioned above.

In the first to fifth embodiments mentioned above, one of the therapeutic energy applying structures 9 (9′) and 9A to 9D is provided on both of the holding members 8 and 8′. Alternatively, one of the therapeutic energy applying structures 9 (9′) and 9A to 9D may be provided on only one of the holding members 8 and 8′.

In the first to fifth embodiments mentioned above, the therapeutic energy applying structures 9 (9′) and 9A to 9D are configured to apply only heat energy to body tissue. Alternatively, the therapeutic energy applying structures 9 (9′) and 9A to 9D may be configured to apply high-frequency energy or ultrasound energy, in addition to the heat energy.

According to some embodiments, it is possible to avoid local overheat of the electric resistance pattern.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A therapeutic energy applying structure, comprising:
an electric resistance pattern configured to generate heat by applying current;
a heat transfer plate configured to transfer the heat from the electric resistance pattern to a body tissue;
an adhesive layer having heat conductivity and provided between the electric resistance pattern and the heat transfer plate to adhesively fix the electric resistance pattern and the heat transfer plate; and
a heat diffusion layer provided between the electric resistance pattern and the adhesive layer and configured to diffuse the heat from the electric resistance pattern and transfer the diffused heat to the adhesive layer, wherein
the adhesive layer is configured to adhesively fix the electric resistance pattern and the heat transfer plate via the heat diffusion layer.

2. The therapeutic energy applying structure according to claim 1, wherein
the heat diffusion layer is a layer that is formed on a surface of the electric resistance pattern by applying energy to materials in a particulate state, a molecular state, or an atomic state.

3. The therapeutic energy applying structure according to claim 1, wherein
thermal conductivity of the heat diffusion layer is higher than thermal conductivity of the adhesive layer.

4. The therapeutic energy applying structure according to claim 1, wherein
thermal resistance per unit cross-sectional area of the heat diffusion layer is smaller than thermal resistance per unit cross-sectional area of the adhesive layer.

5. The therapeutic energy applying structure according to claim 4, wherein
a relationship between the thermal resistance per unit cross-sectional area of the heat diffusion layer and the thermal resistance per unit cross-sectional area of the adhesive layer is given by T1/α1<T2/α2 where a thickness of the heat diffusion layer is T1, thermal conductivity of the heat diffusion layer is α1, a thickness of the adhesive layer is T2, and thermal conductivity of the adhesive layer is α2.

6. The therapeutic energy applying structure according to claim 1, wherein
the heat diffusion layer comprises an insulating layer and a heat conducting layer that are provided independently of each other, and
the insulating layer is located closer to the electric resistance pattern than the heat conducting layer.

7. The therapeutic energy applying structure according to claim 1, wherein
a coefficient of thermal expansion of the heat diffusion layer is a value closer to a coefficient of thermal expansion of the electric resistance pattern than a coefficient of thermal expansion of the adhesive layer.

8. A medical treatment device comprising the therapeutic energy applying structure according to claim 1.
